(19) United States
(12) Patent Application Publication
(10) Pub. No.: US 2011/0019893 A1
(43) Pub. Date: Jan. 27, 2011

(54) Method and Device for Controlling the Ablation Energy for Performing an Electrophysiological Catheter Application

(76) Inventors: Norbert Rahn, Forchheim (DE); Dietrich Till, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/840,716

(22) Filed: Jul. 21, 2010

(30) Foreign Application Priority Data
Jul. 22, 2009 (DE) 10 2009 034 249.4

(51) Int. Cl. G06K 9/00; A61B 18/18

(52) U.S. Cl. 382/131; 606/41

(57) ABSTRACT

A device and a method for controlling ablation energy for performing an electrophysiological catheter application are provided. Measured parameters that are characteristic for guidance of a catheter are received by a communication module. The characteristic parameter values are compared with at least one predefined threshold value by a control module. The control module generates control data for guidance of the catheter as a function of the result of the comparison. The control data is output to at least one control station by output interfaces for controlling the guidance of the catheter for the purpose of adjusting the ablation energy of the catheter.

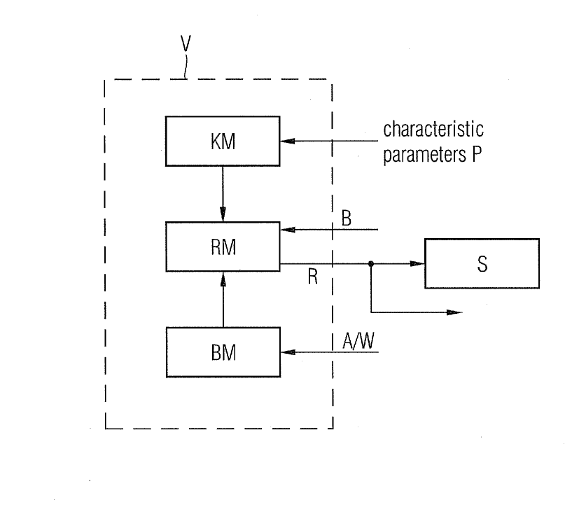

### CROSS REFERENCE TO RELATED APPLICATIONS

This application claims priority of German application No. 10 2009 034 249.4 filed Jul. 22, 2009, which is incorporated by reference herein in its entirety.

### FIELD OF THE INVENTION

The invention relates to a method and a device for visually supporting an electrophysiological catheter application according to the respective preambles of the independent claims.

### BACKGROUND OF THE INVENTION

The treatment of heart rhythm abnormalities (cardiac arrythmias) has evolved significantly since the introduction of the technique of catheter ablation by means of high-frequency current. With this technology an ablation catheter is introduced via veins or arteries into one of the ventricles of the heart under X-ray control and the tissue causing the abnormal heart rhythms is obliterated by means of the application of radiofrequency current. Ablation procedures, e.g. in the left atrium, for treatment of atrial fibrillation, are performed in accordance with electrophysiological and anatomical criteria. In this case three-dimensional morphological information is obtained from imaging modalities such as CT, MR or 3D rotational X-ray angiography, such as is known e.g. from DE 10 2005 016 472 A1.

A prerequisite for successfully performing a catheter ablation is the precise localization of the cause of the cardiac arrhythmia in the ventricle. Said localization is typically accomplished by way of an electrophysiological investigation in which electrical potentials are recorded in a spatially resolved manner by means of a mapping catheter introduced into the ventricle. Accordingly, 3D mapping data is obtained from said electrophysiological investigation, referred to as electroanatomical mapping or imaging, and said data can be visualized on a monitor. In many cases the mapping function and the ablation function are therein combined in one catheter, such that the mapping catheter simultaneously serves also as an ablation catheter.

The following electroanatomical tracking or 3D mapping methods are possible:

The Carto system from the company Biosense Webster Inc., USA can import and segment three-dimensional morphological image data and overlay said data with the electroanatomical mapping data. In this case anatomical landmark pairs are typically used which are identified both in the mapping data and in the 3D image data and then used to produce the overlay. Furthermore the surface of the Carto model can be overlaid with the 3D image data by surface registration, as is known for example from DE 103 40 544 B4.

The NavX system from St. Jude Medical can import and segment three-dimensional morphological image data and overlay said data with the electroanatomical mapping data. In this case anatomical landmark pairs are used which are identified both in the mapping data and in the 3D image data and then used to produce the overlay. An enhanced registration method compared with that described above is possible in this case.

The TactiCath catheter (Enclosense, Meyrin, Switzerland) is conceivable as a catheter which enables the contact force on the endocardium of the heart ventricle that is to be ablated to be measured and said measurement data to be made available as external information.

The objective here is to perform the therapy as effectively as possible on the basis of the three-dimensional morphology.

The effectiveness of an ablation lesion (e.g. transmurality) at each ablation point is dependent on
the local anatomical properties of the target tissue (tissue strength, risk factor of the target region)
local contact pressure (contact force) of the ablation catheter on the myocardium
energy (power) delivered by the ablator
ablation duration (local dwell time) at an ablation point

Currently, these parameters are varied intuitively by manual parameterization of the ablator (e.g. setting of maximum values) and by means of the catheter guidance, without the dependencies of the parameters (contact pressure, dwell time, anatomy) being taken into consideration. The parameters vary greatly in a user-specific manner. The same applies to the anatomy of the patient.

The result are ablation lesions of different efficacy (e.g. interrupted instead of—as desired—uninterrupted ablation lines) which possibly do not lead to the desired success of the therapy and necessitate the repetition of the entire procedure at a later time.

### SUMMARY OF THE INVENTION

The object of the present invention consists in disclosing a method and a device for controlling or monitoring a catheter ablation which enable an improved orientation of the guidance of the catheter and improved catheter application.

The object is achieved by means of the method and the device as claimed in the independent claims. Advantageous embodiments of the method and of the device are the subject matter of the dependent claims or may be derived from the following description as well as from the exemplary embodiments.

The subject matter of the invention is an automatically controlled ablation system in the form of a method or device which produces the optimal lesion by controlling the delivery of the ablation energy taking into account the parameters

contact pressure of the ablation catheter

dwell time (ablation duration at an ablation point)

individual morphological properties of the target region.

The invention describes an ablation system which produces the optimal lesion by controlling the delivery of ablation energy taking into account the parameters

contact pressure of the ablation catheter

ablation duration at an ablation point

morphological properties at the current ablation point.

This results in effective ablation lesions which increase the success rate of the ablation and reduce the re-ablation rate.

A positive effect on patient safety is also achieved in this case since on the one hand care is exercised with regard to the anatomical risk regions during the therapy, and on the other hand repetitions of the procedure are avoided owing to the increased efficiency of the intervention.

### BRIEF DESCRIPTION OF THE DRAWINGS

Further advantages, details and developments of the invention will emerge from the following description of exemplary embodiments in conjunction with the drawings, in which: The FIGURE shows an exemplary schematic representation of the invention.

### DETAILED DESCRIPTION OF THE INVENTION

In this regard the FIGURE shows the individual steps in the performance of the method according to the invention or, as the case may be, the individual modules of the associated device V.

The invention describes an ablation system in which the energy delivered by the ablator is set or adjusted (automatically) based on parameters P such as e.g. “catheter contact pressure” and “ablation duration” on the basis of (location-dependent) knowledge of the intended ablation lesion.

It is favorable to generate an ideal lesion for each ablation point—taking into account the interaction (e.g. weighted sum) of the three cited parameters “catheter contact pressure”, “energy delivery of the ablator” and “local ablation duration”, which ideally is predefined by means of a model for the anatomy that is to be treated.

Optional: There exists an anatomical 3D model—such as is described in the foregoing, also called the Atlas model—of the region of interest e.g. heart ventricle in the left atrium
In the Atlas-based model, energy target values are stored at all points (e.g. 0 for risk areas such as pulmonary veins, mitral valve, e.g. 1 at planned lesions or thicker myocardial wall areas).
These target values can be changed by the user e.g. via a user interface B at any time (before the procedure or during the procedure).
The energy target values can also be color-coded (e.g.: green: effective ablation should be possible here; red: here is a risk region at which no ablation is allowed).
The energy target values can also be significantly higher in the immediate environment of previously planned ablation lesions than in regions that are more remotely located relative to the planned lesions.
Together with the energy target values the following are stored for each site:
   Minimum/maximum for the catheter contact pressure (vertical angle of the catheter to the endocardium is assumed)
   Minimum/maximum or the ablation energy
   Minimum/maximum or the ablation duration
   (for example weighted) sum of the three last-cited parameters
   Automatic control of the ablator is accomplished based on an algorithm which automatically adjusts the critical parameters to the respective anatomical requirements during the application, e.g. correctively adjusts time or energy as a function of the catheter contact pressure. For this purpose the ablation system described here has a hardware interface having a communication protocol between ablator and unit for measuring the catheter contact pressure.

There exists a 3D image data set or an anatomical model of the heart ventricle that is to be treated, said anatomical model having been produced as a result of electroanatomical mapping.

The spatial position (and three orientations) of the ablation catheter is continuously available.

A system which measures the contact pressure of the ablation catheter on the endocardium is available. The system also furnishes information indicating whether the tip of the catheter or a side of the catheter is touching the endocardium.

All control data required for controlling the ablator is automatically transmitted in near real-time to the ablator (or a system S that controls the ablator).

Prior to the ablation procedure: The Atlas-based 3D model is adapted to the 3D image data (matching with deformation of the Atlas-based 3D model). Alternatively, three-dimensional electroanatomical mapping data can also be used instead of the 3D image data. During the ablation procedure the ablation catheter can be assigned (because its position is known) to a point of the Atlas model, as a result of which the target values of the ablation are predefined for each ablation point.

The Atlas model can also contain anatomical lesion plans which can be dynamically updated by the user prior to or during the procedure.

The ablation system has the following characteristics, the location-dependent entries being used in the Atlas model:

Control is implemented via an interface between contact pressure sensor and ablator. Thus, in the event of higher contact pressure the energy delivery of the ablator is automatically reduced to a predefined threshold value—input, where appropriate, via a user interface B.

According to the invention, measured parameters P that are characteristic for guidance of a catheter are received by a contact pressure sensor in a communication module KM. The characteristic parameters P preferably include as values catheter contact pressure, ablation energy and ablation duration. The communication module should also take into account the characteristics of the currently employed catheter type, such as e.g. form of energy delivery (unipolar/bipolar), catheter tip length/diameter, no/open-loop/closed-loop irrigation of the catheter tip. It would be conceivable to store said characteristics with the Atlas model as a “setup”.

Even with a longer dwell duration of the catheter at a location the energy delivery of the ablator is continuously reduced (as a function of the contact pressure of the ablation catheter). The user or investigator is kept informed about the automatically modified energy delivery: This can be done via an acoustic output and/or display element in the UI (User Interface) of the ablation system (e.g. bar which color-codes the energy or simply numeric output of the energy) or alternatively or in addition via acoustic output of a tone whose volume and/or pitch represent(s) the amplitude of the delivered energy.

The energy delivery of the ablator is stopped if e.g. the weighted sum of the characteristic parameter values P contact pressure, energy and dwell duration exceeds the threshold value predefined in the Atlas model. This is preferably performed in a control module RM which generates control data R for the purpose of catheter guidance as a function of the result of the comparison and outputs the control data to at least one control station S controlling the catheter guidance via one or more output interfaces.

Optional (according to the invention, option can be switched on and off): The energy delivery of the ablator is controlled as a function of the current distance/spacing A of the ablation catheter tip from the previously planned lesion (which—as described above—is stored in the 3D Atlas model). Accordingly, the maximum energy (taking into account the parameters contact pressure, energy and dwell duration) is delivered exclusively in the immediate vicinity of the planned lesion (therapy region), reducing to a minimum value as the distance from the planned lesion increases. In this case the relation between “distance from planned lesion” and “reduction in energy delivery” can be configured via a—not necessarily linear—look-up table. This is preferably performed and controlled by a calculation module BM.

With regard to the parameter “contact pressure of the ablation catheter”, the two solid angles W of the catheter tip relative to the endocardial wall are also taken into account (the angles are measured by means of pressure sensors at the catheter tip and on the side of the catheter). Thus, a stronger wall contact is assumed if the angle is more vertical than if the angle is flatter. More vertical angles therefore result in an increase in the parameter values, whereas flatter angles result in a reduction in the parameter values.

If an active navigation system e.g. S is used for the ablation, in addition or alternatively to the variation of the energy delivery the contact pressure or the position of the ablation catheter can also be automatically changed (e.g. reduced).

Visualization of the parameters e.g. on a display device (not shown):

Each ablation point is entered in the 3D model. Color coding of the ablation point is carried out based on the (for example weighted) sum of the parameters contact pressure of the ablation catheter, ablation energy, ablation duration. Thus, for example, the ablation point is initially green at the commencement of the ablation and changes its color continuously until the energy target value of said location has been reached (the point is then colored red, for example).

During the ablation the three parameters contact pressure, energy delivery, dwell duration are displayed in the UI of the ablation system. Bars whose length indicates the amplitude of the parameters can serve as indicators, for example. The bars can also be color-coded (e.g. on the basis of the specifications stored in the Atlas model in relation to minimum/maximum of the three parameters). Thus, for example, each of the three bars can be green if the parameter at the ablation site lies within the defined interval, and change to red as soon as the interval is left.

The combination of the three parameters can be displayed in a similar manner via a fourth bar.

The ablation system described here can also operate in a simplified variant, such as e.g. without the information furnished by the Atlas model: In said simplified variant a location-constant threshold value is predefined which is not to be exceeded by the weighted sum of contact pressure, ablation energy and ablation duration (that is achieved—as described above—through control of the ablator).

1.-16. (canceled)

17. A device for controlling an ablation energy when performing an electrophysiological catheter application, comprising:
a communication module that receives a characteristic parameter for guidance of a catheter;
a control module that compares the characteristic parameter with a predefined threshold value and generates a control data for the guidance of the catheter based on the comparison; and
an output interface that outputs the control data to a control station for controlling the guidance of the catheter and for adjusting the ablation energy of the catheter.

18. The device as claimed in claim 17, further comprising an input interface that receives an electroanatomical 3D mapping data and/or a 3D image data extracted in a region of an interest for overlaying with the 3D mapping data.

19. The device as claimed in claim 17, further comprising a display device that represents the control data visually or acoustically.

20. The device as claimed in claim 17, wherein the control module comprises a graphical user interface for an operator to manually specify the threshold value.

21. The device as claimed in claim 17, further comprising a calculation module that calculates a current distance of a catheter tip to a predefinable image point in the 3D image data and/or the 3D mapping data and stores the distance in the control data.

22. The device as claimed in claim 17, further comprising a calculation module that calculates a current angle of a catheter tip relative to a predefinable image point in the 3D image data and/or the 3D mapping data and stores the angle in the control data.

23. A method for controlling an ablation energy when performing an electrophysiological catheter application, comprising:
measuring a characteristic parameter for guidance of a catheter during the catheter application;
comparing the characteristic parameter with a predefined threshold value;
generating a control data for the guidance of the catheter based on the comparison; and
outputting the control data to a control station for controlling the guidance of the catheter and for adjusting the ablation energy of the catheter.

24. The method as claimed in claim 23, further comprising:
providing an electroanatomical 3D mapping data of a region of interest, and/or
acquiring a 3D image data of the region of interest by a 3D imaging device prior to the catheter application, and
segmenting the 3D image data for extracting a 3D surface profile data of an object in the region of interest.

25. The method as claimed in claim 24, wherein the 3D image data is acquired by an X-ray computed tomography device, a magnetic resonance tomography device, or a 3D ultrasound device.

26. The method as claimed in claim 24, wherein the control data is integrally represented in an overlaid visualization of the 3D mapping data with the extracted 3D surface profile data.

27. The method as claimed in claim 23, wherein the control data is represented visually or acoustically.

28. The method as claimed in claim 23, wherein the characteristic parameter comprises values of catheter contact pressure, ablation energy, and ablation duration.

29. The method as claimed in claim 23, wherein a weighted sum is calculated from the values of catheter contact pressure, ablation energy, and ablation duration and is compared with the threshold value.

30. The method as claimed in claim 23, wherein the threshold value comprises an interval in a maximum value and a minimum value.

31. The method as claimed in claim 23, wherein a current distance of a catheter tip relative to a predefinable image point in the 3D image data and/or the 3D mapping data is calculated and is stored in the control data.

32. The method as claimed in claim 23, wherein a current angle of a catheter tip relative to a predefinable image point in the 3D image data and/or the 3D mapping data is calculated and is stored in the control data.
